(19) United States
(12) Patent Application Publication
(10) Pub. No.: US 2022/0322942 A1
(43) Pub. Date: Oct. 13, 2022

(54) MICROMOTOR-INTEGRATED ENDOSCOPIC SIDE-VIEWING PROBE

(71) Applicant: The University of British Columbia, Vancouver (CA)

(72) Inventors: Kenichi TAKAHATA, Vancouver (CA); Babak ASSADSANGABI, Vancouver (CA); Sayed Mohammad Hashem JAYHOONI, Vancouver (CA)

(21) Appl. No.: 17/843,561

(22) Filed: Jun. 17, 2022

Related U.S. Application Data
(63) Continuation of application No. PCT/CA2020/051770, filed on Dec. 18, 2020.
(60) Provisional application No. 62/950,846, filed on Dec. 19, 2019.

(51) Int. Cl. A61B 5/00; H02K 7/09; A61B 1/00

(52) U.S. Cl. CPC A61B 5/0062; H02K 7/09; A61B 1/00167

(57) ABSTRACT

An endoscopic probe comprises a flexible light guide extending from a proximal end of the endoscopic probe to a distal end portion of the endoscopic probe. A motor is disposed in the distal end portion of the endoscopic probe. The motor comprises a rotor coupled to drive rotation of a light deflector. The light deflector is located between the rotor and a distal end of the endoscopic probe. The rotor is configured to provide a light path extending axially through the rotor. The light path arranged to carry light between the light deflector and the light guide. The endoscopic probe may be applied for helical scanning walls of small passages in any of a wide range of modalities such as OCT, fluorescence imaging, Raman spectroscopy, reflectance imaging.

### CROSS-REFERENCE TO RELATED APPLICATION

This application is a continuation of Patent Cooperation Treaty (PCT) application No. PCT/CA2020/051770 having an international filing date of 18 Dec. 2020 and entitled MICROMOTOR-INTEGRATED ENDOSCOPIC SIDE-VIEWING PROBE, which in turn claims priority from, and the benefit under 35 U.S.C. § 119 of, U.S. application No. 62/950,846 filed 19 Dec. 2019 and entitled MICROMOTOR-INTEGRATED ENDOSCOPIC SIDE-VIEWING PROBE. All of the applications referred to in this paragraph are hereby incorporated herein by reference for all purposes.

### TECHNICAL FIELD

The present invention relates to side-viewing endoscopic probe devices employing micromotors. Such devices have example applications in soft tissue imaging.

### BACKGROUND

Endoscopic devices can be used for diagnosis and identification of tumors and cancer tissues in tubular ducts in vivo. In some important applications an endoscopic probe is passed through curved and confined internal body channels. In such applications an endoscopic probe should be flexible and manoeuverable to enable repeatable and precise positioning without damaging tissue.

In microendoscopic applications, an endoscopic probe should be small enough to safely reach a target in vivo location through very narrow channels. For example, the lumen of bronchi in the lungs may have internal diameters of less than 3 mm.

Forward-viewing microendoscopic probes are commercially available. However, such microendoscopic probes are limited to viewing tissues located in front of the endoscopic probe (e.g. tissue at the end of a duct or where the duct bends). Side-viewing probes which use rotary micromotors to scan a viewing direction have been developed. In such a probe a micromotor may be coupled to drive rotation of a mirror to circumferentially probe the interior tissue wall.

Motor driven microendoscopic devices can be limited by the large dimensions of motors. Also, designs which incorporate motors can require a long rigid portion in which the micromotor and imaging components are enclosed. A long rigid part at the distal end of the catheter can make the catheter difficult to manoeuver in curved ducts. For example, Yang et. al, “Photoacoustic endoscopy,” Opt. Lett. 34, 1591-1593 (2009) discloses a photoacoustic endoscopy catheter with 5 cm of rigid length.

There is a general desire for improved microendoscopic devices which may be used in biological imaging applications. There is a need for such devices to be more maneuverable within a subject's organs.

The foregoing examples of the related art and limitations related thereto are intended to be illustrative and not exclusive. Other limitations of the related art will become apparent to those of skill in the art upon a reading of the specification and a study of the drawings.

### SUMMARY

This invention has a number of aspects that may have synergy when combined but also have application individually. These aspects relate to constructions for endoscopic probes and methods for making endoscopic probes.

One aspect of the invention provides an endoscopic probe comprising a flexible light guide extending from a proximal end of the endoscopic probe to a distal end portion of the endoscopic probe. A motor is provided in the distal end portion of the endoscopic probe. The motor comprises a rotor that is rotatable relative to the light guide. A light deflector is connected to be driven to rotate by the rotor. The light deflector is located between the rotor and a distal end of the endoscopic probe. The rotor is configured to provide a light path extending axially through the rotor, the light path arranged to carry light between the light deflector and the light guide.

In some embodiments the light path comprises an aperture that extends axially into the rotor. The aperture may extend axially through the rotor. In some embodiments the light guide extends into the aperture. A distal end of the light guide may be on a distal side of the rotor between the rotor and the light deflector. In some embodiments an interior wall of the aperture is reflective. For example, a reflective layer may be disposed on the interior wall of the aperture or the interior wall of the aperture may be polished.

In some embodiments the light guide extends into the rotor along the light path. For example, the light guide may extend completely through the rotor and terminate between the rotor and the light deflector.

In some embodiments the endoscopic probe comprises a cantilever member extending into the aperture and an electronic device supported by the cantilever member. The electronic device may comprise one or more light sources. one or more ultrasound transducers, one or more light sensors, and/or a camera comprising a 2D array of pixels. In some embodiments at least part of the electronic device is located in the aperture of the rotor. In some embodiments the electronic device is located distally of the rotor between the rotor and the light deflector. In some embodiments the endoscopic probe includes a cable extending along the endoscopic probe and the cable comprises at least one conductor extending along the cantilever member and connected to the electronic device.

In some embodiments the endoscopic probe comprises a lens supported in the light path to rotate with the rotor. For example, the lens may be a GRIN lens.

In some embodiments the endoscopic probe comprises a light guide in the light path and coupled to rotate with the rotor and/or an optically transparent window carried on the rotor.

In some embodiments the light deflector is surrounded by a 360 degree unobstructed window.

In some embodiments the light deflector comprises a mirror or a prism.

In some embodiments the light deflector is coupled to be rotated by the rotor by magnetic interaction between the rotor and a magnet attached to the light deflector. In some embodiments the light deflector is coupled to be rotated by the rotor by a member that attaches the light deflector to the rotor.

Some embodiments provide a micro-lens disposed at a distal end of the light guide.

In some embodiments the rotor comprises a magnet. For example, the magnet may be a rare earth magnet. The magnet may have a rectangular (e.g. square) cross section. For example, the magnet may be cube shaped. In some embodiments the magnet has a cross sectional shape that includes corners at circumferentially spaced apart locations. The corners may lie on a common circle. In some embodiments the rotor comprises a plurality of magnets connected together. For example, the plurality of magnets may be spaced apart by a transparent material. In an example embodiment the rotor consists of a rare earth magnet having a rectangular cross section, with north and south magnetic poles on first and second opposed faces of the magnet and a hole extending axially between the first and second faces through the rare earth magnet from a proximal end face of the magnet to a distal end face of the magnet wherein the light path extends along the hole.

In some embodiments the endoscopic probe comprises a fluidic channel extending from the proximal end of the endoscopic probe to a chamber located adjacent to the rotor. The rotor may be supported for axial movement relative to the light guide and an axial position of the light deflector may be adjustable by introducing a fluid into the chamber by way of the fluidic channel or withdrawing the fluid from the chamber by way of the fluidic channel. In some embodiments the motor comprises a ring located proximally relative to the rotor and a coupling connecting the ring to the rotor, the coupling operable to transmit axial and rotational motion between the ring and the rotor.

In some embodiments the rotor is radially supported in an interior of a tubular motor housing by one or more bearings. When the rotor is magnetic the one or more bearings may comprise ferrofluid bearings. In some embodiments one or more stoppers are arranged to axially support the rotor. The one or more stoppers may comprise sealing media operable to maintain an axial position of the one or more bearings. In some embodiments the one or more stoppers comprise garter springs.

In some embodiments the endoscopic probe comprises a flexible tubular sheath, the tubular sheath made at least in part from fluorinated ethylene propylene.

In some embodiments the rotor is located in a bore of a tubular motor housing. The tubular motor housing may, for example comprise a heat-shrinkable tube, the heat shrinkable tube radially surrounding the motor and the light guide.

In some embodiments the endoscopic probe comprises plural stator conductors extending along the endoscopic probe toward the distal end of the endoscopic probe, the stator conductors extending past the rotor in a generally axial direction at locations circumferentially spaced apart around the rotor. The stator conductors are optionally stacked at selected circumferential locations around the rotor. In some embodiments the light deflector is located distally of a farthest distal extent of the stator conductors. In some embodiments portions of the stator conductors near the light deflector are transparent (e.g. made from a conductive transparent material such as indium tin oxide. The stator conductors may extend along an inner wall of a tubular sheath of the endoscopic probe.

Portions of the stator conductors may be provided by traces on a flexible printed circuit sheet that is flexed to curve around the rotor. The flexible printed circuit sheet may be attached to an inner wall of a tubular member.

In some embodiments a diameter of the motor is 2 mm or less.

In some embodiments an outside diameter in a section of the endoscopic probe extending from the distal end is for a distance of at least 50 cm toward the proximal end is 2 mm or less.

Another example aspect of the invention provides a method for manufacturing an endoscopic probe. The method comprises: depositing one or more stator conductors onto a flexible sheet to form a stator sheet; inserting the stator sheet into a heat-shrinkable tube; inserting a rigid rod into the heat-shrinkable tube, the rigid rod having a diameter that provides a desired radial dimension of the endoscopic probe; heating the heat-shrinkable tube containing the stator sheet, thereby shrinking the heat shrinkable tube to have the desired radial dimension of the endoscopic probe; removing the rigid rod from the heat-shrinkable tube; and inserting a light guide into one end of the heat-shrinkable tube and inserting a motor into an opposite end of the heat-shrinkable tube.

In some embodiments the heat-shrinkable tube is made at least in part from fluorinated ethylene propylene.

Another example aspect of the invention provides a method for manufacturing an endoscopic probe. The method comprises: depositing one or more stator conductors onto a flexible sheet to form a stator sheet; inserting the stator sheet into a heat-shrinkable tube; positioning the heat-shrinkable tube around a rotor that is coated with a removable outer layer; heating the heat-shrinkable tube containing the stator sheet and the rotor to cause the heat-shrinkable tube to shrink around the removable outer layer on the rotor; removing the removable outer layer; and depositing a ferrofluid bearing in a space between an inner wall of the heat-shrunk tube and an outer wall of the rotor.

In addition to the exemplary aspects and embodiments described above, further aspects and embodiments will become apparent by reference to the drawings and by study of the following detailed descriptions.

### BRIEF DESCRIPTION OF THE DRAWINGS

Example embodiments are illustrated in the enclosed drawings. It is intended that the embodiments and figures disclosed herein are to be considered illustrative rather than restrictive.

FIG. 1A is a schematic longitudinal cross-sectional drawing of a distal end portion of an example endoscopic probe using a magnetic micromotor. FIG. 1B is a cross section view of the endoscopic probe on the lines 1B-1B of FIG. 1A. FIGS. 10, 1D and 1E are schematic drawings showing examples of flexible sheets of material carrying electrically conductive stator conductors in an unrolled state. FIG. 1F illustrates example activation logic of stator conductors for driving the rotation of a magnetic rotor. FIG. 1G schematically illustrates a light deflector mounted at an angle to a rotor.

FIG. 2A is a schematic longitudinal cross-sectional drawing of a distal end portion of an example endoscopic probe having light guides and an inserted sealing tube. FIG. 2B is a schematic longitudinal cross-sectional drawing of a distal end portion of an example endoscopic probe having a flipped light deflector held by a light deflector holder. FIG. 2C is schematic longitudinal cross-sectional drawing of a distal end portion of an example endoscopic probe having light guides extending inside a magnetic rotor, the magnetic rotor being attached to a light deflector.

FIG. 3 is a schematic longitudinal cross-sectional drawing of a distal end portion of an example endoscopic probe having a light guide capable of receiving and transmitting light in the same portions of the light guide.

FIG. 4 is a schematic longitudinal cross-sectional drawing of a distal end portion of an example endoscopic probe having light guides axially spaced from a magnetic rotor.

FIG. 5 is a schematic longitudinal cross-sectional drawing of a distal end portion of an example endoscopic probe having light guides axially spaced from an apertured magnetic rotor having interior reflective surfaces or interior surfaces lined with a reflective coating.

FIG. 6 is a schematic longitudinal cross-sectional drawing of a distal end portion of an example endoscopic probe having light guides and a collimator axially spaced from a magnetic rotor.

FIG. 7A is a schematic longitudinal cross-sectional drawing of a distal end portion of an example endoscopic probe having a magnetic rotor comprising two magnets. FIG. 7B is a cross-section view on the lines 7B-7B of FIG. 7A.

FIG. 8 is a schematic longitudinal cross-sectional drawing of a distal end portion of an example endoscopic probe incorporating a GRIN lens.

FIG. 9 is a schematic longitudinal cross-sectional drawing of a distal end portion of an example endoscopic probe having two axially spaced magnets.

FIG. 10 is a schematic longitudinal cross-sectional drawing of a distal end portion of an example endoscopic probe having two axially spaced magnets and an ultrasound transducer.

FIG. 11 is a schematic longitudinal cross-sectional drawing of a distal end portion of an example endoscopic probe having two axially spaced magnets and an ultrasound transducer for transmitting and receiving ultrasound waves.

FIG. 12 is a schematic longitudinal cross-sectional drawing of a distal end portion of an example endoscopic probe having an ultrasound transducer for transmitting and receiving ultrasound waves.

FIG. 13 is a schematic longitudinal cross-sectional drawing of a distal end portion of an example endoscopic probe having an image sensor disposed near a distal end of a magnetic rotor.

FIG. 14 is a schematic longitudinal cross-sectional drawing of a distal end portion of an example endoscopic probe having a proximally disposed image sensor and a distally disposed lens.

FIG. 15 is a schematic longitudinal cross-sectional drawing of a distal end portion of an example endoscopic probe having means for longitudinally positioning a magnetic rotor.

FIG. 16A is a schematic longitudinal cross-sectional drawing of a distal end portion of an example endoscopic probe having a planar stator conductor. FIG. 16B is a cross section view of an expanded planar stator conductor in the plane indicated by lines 16B-16B of FIG. 16A. FIG. 16C is a schematic view of the flow of electrical current and the resultant magnetic fields in a cross section view of the planar stator conductor of FIGS. 16A and 16B.

FIG. 17 is a schematic view of a system comprising an example endoscopic probe together with supporting systems including a light source, motor driver, control system, and data processing system.

FIG. 18 is a block diagram showing an example method for fabricating endoscopic probes.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

The present technology relates to side-viewing endoscopic probes that include motors for circumferentially scanning a viewing direction around the endoscopic probes. In some embodiments, the probes provide unobstructed viewing while the viewing direction is swept through 360 degrees.

Probes may use different forms of energy for imaging or analysis. For example, a probe as described herein may use optical energy (e.g. visible light, infrared light) or ultrasound energy for imaging.

In some embodiments, a probe includes a motor that has a rotor configured with an axial window or aperture and a deflector. The rotor drives rotation of the deflector. The deflector directs imaging energy from the probe in an imaging direction and directs returning energy toward the axial window or aperture in the rotor.

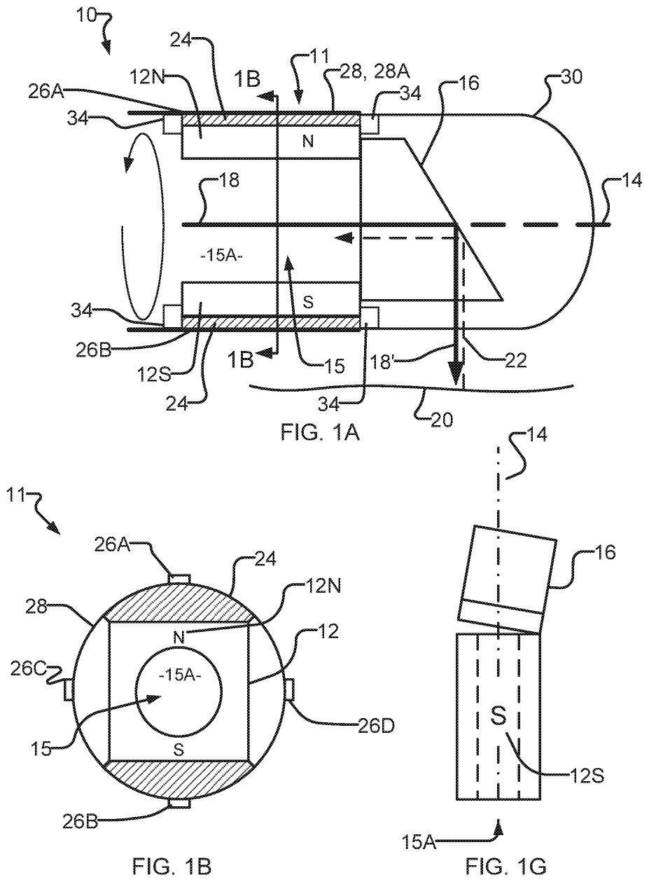

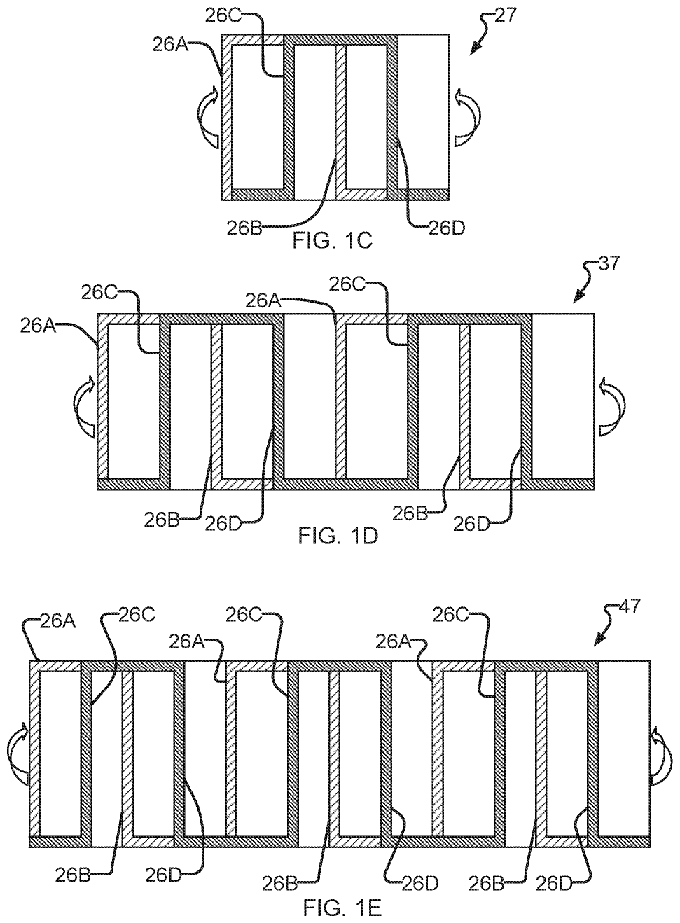

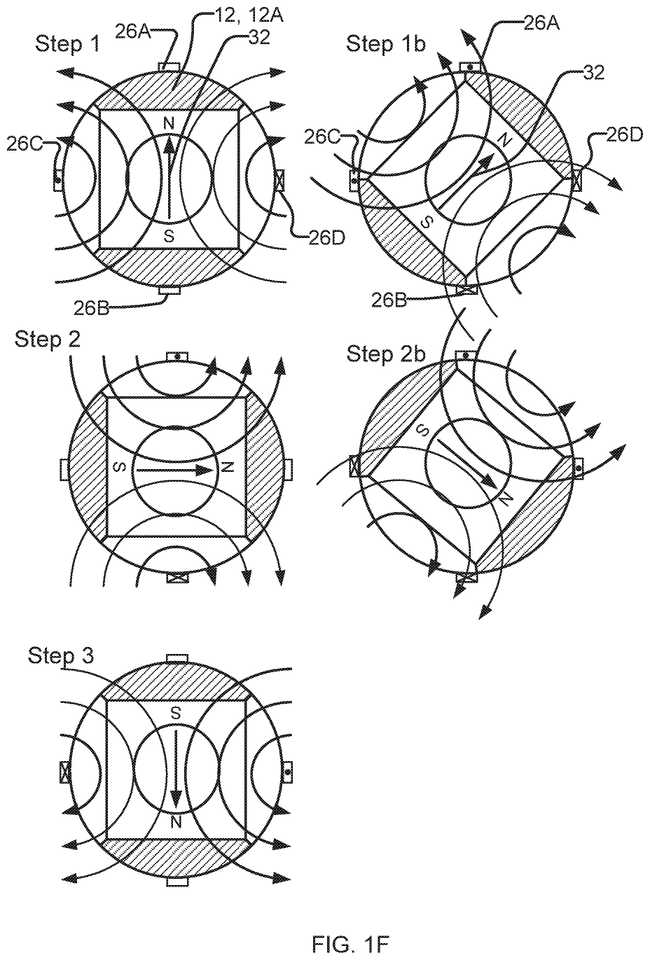

In example embodiments where the imaging energy is a form of light, the deflector is a light deflector (e.g. a prism or mirror) arranged to receive light incident in a viewing direction from surrounding tissues and to deflect the received light to pass through the rotor and/or to receive light from an illumination system that has passed through the rotor and deflect the light received from the illumination system to illuminate the tissues. FIGS. 1A to 9 illustrate various example embodiments which use light as imaging energy.

FIG. 1A is a schematic longitudinal cross-section of the distal end of an example endoscopic probe 10 which may be applied to scan tubular ducts within human or animal organs. Endoscopic probe 10 has a proximal end that can be connected to deliver light to a light detection system (not shown in FIG. 1A) and a distal end that can be inserted into a passage within a patient. Endoscopic probe 10 typically has a length between the proximal and distal ends in the range of about 40 cm to 1 m. Between its proximal and distal ends, endoscopic probe 10 is typically flexible and sized to allow endoscopic probe 10 to be threaded into small passages such as peripheral airways of the lung.

Endoscopic probe 10 comprises a motor 11 comprising a magnetic rotor 12. Magnetic rotor 12 is magnetized and has angularly spaced apart magnetic poles 12N and 12S. Pole(s) 12N are north poles and pole(s) 12S are south poles of rotor 12. The illustrated rotor has two magnetic poles. However rotor 12 could be constructed to have a larger number of radially facing magnetic poles.

Rotor 12 may, for example, comprise a rare earth magnet such as a neodymium magnet or a samarium-cobalt magnet or a cobalt-platinum alloy magnet. Rotor 12 may, for example comprise a rare earth magnet in the form of a cube or a square prism having an opening extending between two end faces and having opposed magnetic poles located on side faces. Rotor 12 may, for example, comprise an electroplated permanent magnet such as a cobalt-platinum alloy magnet.

Rotor 12 is supported to rotate about a longitudinal axis 14. Longitudinal axis 14 may be coaxial with endoscopic probe 10. A light deflector 16 is coupled to be driven to rotate about axis 14 by rotor 12. Light deflector 16 may, for example comprise a mirror, prism, light pipe, or other optical element that deflects light travelling in either direction between an on-axis direction generally parallel to axis 14 and a deflected direction that is non-parallel to axis 14. It is not necessary for the deflected direction to be perpendicular to the on-axis direction. In some embodiments the angle between the on-axis direction and the deflected direction is in the range of about 60 degrees to about 140 degrees.

Light deflector 16 is connected to rotor 12 at a distal end of rotor 12. Light deflector 16 may be connected to rotor 12 in any suitable way. For example, light deflector 16 may be:
bonded to rotor 12 using any appropriate adhesive,
connected to rotor 12 by an appropriate mechanical coupling, or
integrally formed with rotor 12 (e.g. rotor 12 and deflector 16 may both be provided by a unitary piece of material).

In some embodiments, an interference fit is employed to provide a mechanical coupling. For example, light deflector 16 may comprise a recess and rotor 12 may comprise a corresponding protrusion (or vice versa) to achieve an interference fit between the recess and protrusion. In other embodiments, torque transmission between light deflector 16 and rotor 12 may be provided through a keyed joint design. In yet another embodiment, light deflector 16 and rotor 12 are connected by an intermediate spacer.

In some embodiments light deflector 16 is tilted at a small angle (e.g. an angle of a few degrees up to about 15 or so degrees) to rotor 12 as illustrated, for example, in FIG. 1G. Mounting light deflector 16 to rotor 12 at a small angle can reduce back reflections of light from interfaces of light deflector 16.

Magnetic rotor 12 is configured to provide a path 15 through which light can pass. Path 15 may be provided by an aperture 15A. Aperture 15A may optionally contain a light guide, a window, a transparent plug, or the like. Path 15 may be coaxial with axis 14. Path 15 may be provided by a hole formed in or made in rotor 12. For example, rotor 12 may comprise a rare earth magnet that has been machined, for example by mechanical drilling or EDM (electrical discharge machining) to provide an opening oriented such that a line between poles 12N and 12S extends transversely relative to the opening.

The amount of magnetic torque generated by rotor 12 should be adequate for rotating light deflector 16 at a desired angular speed, accounting for friction and other sources of energy dissipation. The amount of magnetic torque generated is dependent at least in part on the amount of current delivered through stator conductors 26, a higher current producing more torque. However, the maximum current that can be applied through stator conductors 26 is limited by the current-carrying capacity of stator conductors 26 and the conductors carrying current to stator conductors 26 as well as the maximum acceptable amount of heat that can be generated within a biological subject.

Another factor that can affect the amount of torque produced by motor 11 is the axial length of rotor 12. A longer rotor 12 will generally produce a larger output torque due to the potential for a higher number of coil turns of stators 26 to produce a stronger magnetic field. However, making rotor 12 longer tends to result in a longer part of endoscopic probe 10 being stiff (non-flexible). A longer rotor 12 tends to require a longer part of endoscopic probe 10 to be stiff, which adversely affects maneuverability of endoscopic probe 10.

Incident light 18 is delivered along endoscopic probe 10 and path 15 where it reaches and is deflected by light deflector 16. Deflected incident light 18′ reaches tissue 20. Deflected incident light 18′ interacts with tissue 20 to yield returning light 22. Returning light 22 may be produced, for example, by elastic scatting, inelastic scattering, reflection and/or fluorescence. Returning light 22 travels to light deflector 16, is deflected by light deflector 16 to travel through path 15 and along endoscopic probe 10 to the proximal end of endoscopic probe 10 where light 22 may be detected and analyzed.

Endoscopic probe 10 comprises any appropriate transmission media through which incident light 18 and returning light 22 can travel. For example, endoscopic probe 10 may comprise one or more optical fibers or other light guides arranged to carry returning light 22 or returning light 22 and incident light 18.

To maintain a desired axial position of rotor 12 and bearing 24 within endoscopic probe 10, probe 10 may include stops or guides which limit axial displacement of rotor 12. The illustrated embodiment comprises mechanical stopper rings 34 disposed both proximally and distally of rotor 12. Stopper rings 34 may comprise any appropriate mechanical component or components which limit the axial motion of rotor 12. For example, stopper rings 34 may comprise a circular spring which exerts radial pressure against the interior wall of housing 28. For example, stopper rings 34 may comprise compression garter springs. In other embodiments, stopper rings 34 are bonded or otherwise semi-permanently attached to the interior wall of housing 28.

Endoscopic probe 10 comprises bearing 24 which supports rotor 12 for rotation about axis 14. The net torque that motor 11 can generate can be increased by supporting rotor 12 in a bearing that provides very low friction. A bearing that provides very low friction also facilitates use of a shorter rotor 12.

Bearing 24 is provided by a ferrofluid in some implementations. In other implementations, bearing 24 may comprise other types of bearing such as microballs or self-lubricating sintered metal bearings.

For example, bearing 24 may comprise a layer of a ferrofluid that is magnetically attracted to rotor 12 and is located between rotor 12 and an inside wall of a housing 28 of motor 11. A ferrofluid bearing has an advantage that the attraction of ferrofluid to the magnetic poles of rotor 12 causes the ferrofluid to be pressurized at locations between the poles 12N, 12S and housing 28. This pressure tends to cause rotor 12 to be supported away from the inner wall of housing 28 so that rotor 12 can rotate about axis 14 with low friction.

Another advantage of a ferrofluid bearing is that the ferrofluid can accommodate rotors of various cross-sectional shapes (e.g. round, square, polygonal, etc.). It is desirable for the ferrofluid used in a ferrofluid bearing to have properties of low viscosity and high saturation magnetization for facilitating the effective operation of magnetic rotor 12 by providing low friction and by having strong magnetic properties. Additionally, a low vapor pressure is desirable for increasing the shelf life of bearing 24.

In embodiments where a ferrofluid bearing is provided, a means for preventing the ferrofluid from flowing past a distal edge of rotor 12 and reaching light deflector 16 may be provided. It is undesirable for ferrofluid to reach light deflector 16 because ferrofluids are generally optically opaque liquids which can inhibit the function of light deflector 16 if they become adhered to the light deflector. In some embodiments, garter springs enclosed in rubber sealing media provide a seal to contain ferrofluid 16 from leaking. In embodiments where an intermediate spacer is interposed between rotor 12 and light deflector 16, the intermediate spacer may comprise one or more grooves or other recesses to collect any leaked ferrofluid so as to prevent ferrofluid from reaching light deflector 16.

Some embodiments include structures for preventing ferrofluid from leaking into light path 15. For example, some embodiments include a separator tube that extends axially into bore 15A of rotor 12. The separator tube may project axially past a proximal end of rotor 12. The separator tube optionally comprises inwardly projecting stopper rings in a bore of the separator tube that help to block ferrofluid from migrating along the bore of the separator tube.

Sizes of different components may be varied depending on parameters such as the size of rotor 12 and the strength of the magnet in rotor 12. In a non-limiting example embodiment, rotor 12 comprises a magnet with a square cross-section with side lengths of 1.0 mm and is positioned within in a housing 28 having an internal diameter of 1.65 mm. In this example, there is a gap between 100 μm and 150 μm between the corners of rotor 12 and the inner wall of housing 28. A 300 μm gap between the middle of each lateral surface of rotor 12 and the inner wall of housing 28 is present in this scenario.

It is desirable to construct motor 11 in a way that allows motor 11 to deliver sufficient torque to rotate rotor 12 and deflector 16 at a desired angular rate while making motor 11 compact. This goal can be assisted by making rotor 12 of a material that is strongly magnetized. The higher the field strength of the magnet of rotor 12, the lesser the required driving power, which results in reduced resistive heating, a desirable trait for the operation of endoscopic probe 10. However, removing material to form an aperture for path 15 tends to reduce the strength of a magnet used for rotor 12. There is a trade-off between making the aperture for path 15 larger to facilitate easier and/or more efficient transfer of light or other energy through rotor 12 on path 15 and making the aperture for path 15 smaller, thereby increasing the magnetic strength of rotor 12.

As light deflector 16 is brought closer to the source of incident light 18, the size of aperture 15A of rotor 12 can be reduced since returning light 22 can be more efficiently collected as there is less opportunity for incident light 18 to be scattered, blocked or attenuated. Advantageously, by collimating incident light 18 and increasing the proximity of deflector 16 to the source of light 18, unwanted loss of light can be reduced or avoided and the size of aperture 15A in rotor 12 can thus be reduced. Therefore, an increased magnetic strength of rotor 12 can be achieved (since rotor 12 can contain more magnetic material for the same outside dimensions) while facilitating efficient transfer of light through aperture 15A.

Additionally, by employing a ferrofluid for bearing 24, the friction force against the rotation of rotor 12 is reduced, allowing for rotation using lesser torque, therefore reducing the strength demands of the permanent magnet of rotor 12.

For some intended applications such as scanning small passages in the airways, it is desirable for motor 11 to have a short axial length and to have a small diameter. In some embodiments, the diameter of motor 11 does not exceed about 2.0 mm. In some such embodiments, the combined axial length of rotor 12 and light deflector 16 may be about 10 mm or less. In some applications, an even smaller motor 11 is desirable. For example, for scanning the peripheral airways, the diameter of motor 11 preferably does not exceed 1.3 mm and the combined axial length of rotor 12 and deflector 16 preferably does not exceed 5 mm.

FIG. 1B is a cross-section view through endoscopic probe 10 in the plane indicated by 1B-1B in FIG. 1A showing motor 11. FIG. 1B shows that rotor 12 can have a rectangular or square cross-section with a hole that provides path 15 centered on a central axis of endoscopic probe 10 (e.g. axis 14). Bearing 24 is disposed between rotor 12 and housing 28.

Housing 28 can be rigid to allow rotor 12 and light deflector 16 to turn without binding. Housing 28 may also support a light transmission medium such as one or more optical fibers in a desired alignment with rotor 12. Housing 28 may, for example comprise a short section of relatively rigid tube. Housing 28 may, for example, be made of a suitable plastic. For example, housing 28 may be made from a thermoset plastic such as polyimide. Housing 28 may optionally form part of a sheath that encapsulates endoscopic probe 10.

Motor 11 comprises a number of stator conductors 26. Conductors 26A and 26B are shown in FIG. 1A. Other embodiments may have other numbers of stator conductors 26. For example, FIG. 1B shows additional stator conductors 26C and 26D. Stator conductors 26A, 26B, 26C, and 26D (which may be referred to herein collectively as stator conductors 26 or generally as stator conductor 26) are shown to be angularly spaced around the circumference of housing 28.

Stator conductors 26 are activatable to produce magnetic fields which drive the rotation of rotor 12. Electrical current delivered to different ones of stator conductors 26 may be periodically turned on and off, increased and decreased in magnitude and/or reversed in polarity to create magnetic fields that interact with the magnetic fields of rotor 12 to cause rotation of rotor 12. Stator conductors 26 each comprise one or more windings of a conductor which produce magnetic fields when electric current is passed through the conductor. Electrical current may be provided from a suitable driver circuit by way of wires or other electrical conductors extending along endoscopic probe 10. In some embodiments, electrical current is carried to stator conductors 26 by way of deposited conductors extending along all or part of the length of endoscopic probe 10. Stator conductors 26 may be provided inside, outside, in, or on a housing 28 that supports rotor 12.

In some embodiments, stator conductors 26 comprise electrically conductive traces patterned on a flexible sheet of material that is wrapped to extend around rotor 12 either inside of or outside of a housing 28. When wrapped, the flexible sheet of material may have a cylindrical configuration. FIG. 10 is a schematic drawing of one example flexible sheet of material 27 in an unrolled state. Sheet 27 carries electrically conductive stator conductors 26A, 26B, 26C and 26D. Sheet 27 may be wrapped to extend around rotor 12.

In some embodiments a flexible sheet of material carrying electrically conductive patterned traces which act as stator conductors is of a length that allows the sheet to wrap around rotor 12 two or more times. This construction allows additional arrangements of stator conductors which may, for example, provide increased magnetic fields which may allow rotor 12 to rotate faster at the same electrical driving current.

FIG. 1D is a schematic drawing illustrating an example flexible sheet of material 37 carrying electrically conductive stator conductors 26A, 26B, 26C and 26D in an unrolled state. In this example, sheet 37 is dimensioned to wrap twice around rotor 12. When sheet 37 is wrapped to extend twice around rotor 12, conductors 26A are close to being radially aligned with one another and preferably overlay one another in a double-stacked configuration. This same double-stacked configuration applies to conductors 26B, 26C and 26D. Electrical current flows in the same direction in each set of double-stacked conductors, thereby creating an increased electro magnetic field strength.

FIG. 1E is a schematic drawing of flexible sheet of material 47 carrying electrically conductive stator conductors 26A, 26B, 26C and 26D in an unrolled state. Sheet 47 is dimensioned to be wrapped three times around rotor 12. When sheet 47 is wrapped to extend three times around rotor 12, conductors 26A, 26B, 26C and 26D are each in a triple-stacked configuration. Electrical current flows in the same direction in each set of triple-stacked conductors, thereby creating an increased electro magnetic field strength.

In a non-limiting example embodiment, pairs of stator conductors 26 are electrically connected in series and electrical current through each pair of stator conductors is controlled. For example, in the implementation of FIG. 1B, conductors 26A and 26B may be connected in series and conductors 26C and 26D may be connected in series.

FIG. 1F shows example activation logic of stator conductors 26 to cause a clockwise 180 degree rotation of rotor 12 around axis 14. Stator conductors 26 are angularly spaced apart around rotor 12 at 90 degree intervals. Stator conductors 26A and 26B are connected in series and stator conductors 26C and 26D are connected in series. The sequence of Step 1, Step 2 and Step 3 in FIG. 1F illustrates activating pairs (26A, 26B are one pair and 26C, 26D are another pair) of stator conductors 26 in an alternating manner to produce magnetic fields which electromagnetically drive rotor 12 to rotate in discrete 90 degree steps. This process may be described as a “single-coil” activation mode, whereby at each step a magnetic field is produced by a current driven through a single pair of stator conductors 26.

In FIG. 1F, a dot in the stator conductor 26 cross-section indicates current flowing out of the page and an X in the stator conductor 26 cross-section indicates current flowing into the page. These currents produce magnetic fields as schematically indicated by the curved arrows.

Operation of the single-coil mode involves four steps to achieve a 360 degree rotation (each step achieving a 90 degree rotation). For example, in Step 1 of FIG. 1F, stator conductor 26C carries a current flowing out of the page, producing a counter-clockwise magnetic field while stator conductor 26D receives a current flowing into the page, producing a clockwise magnetic field. The net magnetic field produced by stator conductors 26 produces a magnetic field which causes a torque on rotor 12.

Although FIG. 1F only shows a 180 degree rotation of rotor 12, it will be understood that the principles described herein may be applied to causing continuous full 360 degree rotation of rotor 12. In successive steps the pair of stator conductors to which the current is applied is switched. In successive activations of the same pair of stator conductors the current is reversed.

In some embodiments, stator conductors 26 are operated in a “dual-coil” activation mode whereby current flowing in the same direction is supplied to a pair of adjacent conductors 26 to produce a combined magnetic field. This is illustrated, for example, in Step 1b whereby pairs of adjacent stator conductors 26A and 26C receive a current flowing out of the page to produce a resultant magnetic field. Similarly, adjacent stator conductors 26B and 26D receive a current flowing into the page to produce a resultant magnetic field. This example configuration produces a net torque on rotor 12 driving north pole 12N of rotor 12 45 degrees clockwise from the position in Step 1. Likewise, Step 2b may be performed to enable more precise control of rotor 12 between Step 2 and Step 3.

In the dual-coil activation mode, the angular position of rotor 12 is defined by the balance of the electromagnetic fields generated by two adjacent stator conductors 26, the strength of each electromagnetic field being proportional to the strength of the supplied current. In some embodiments, the exact position of the rotor is controlled by individually changing the amount of current passing through each stator conductor 26 in a synchronized manner. Example methods for controlling the amount of current passing through each stator conductor 26 include varying a level of DC current and varying the pulse duration of a pulsed current (i.e. a pulse width modulation scheme). It is advantageous to employ a dual-coil mode for enabling precision intermediate steps between two adjacent stator conductors 26. The synchronized control of the dual-coil mode permits fine stepping of rotor 12, which may cause rotor 12 to rotate more smoothly and may increase the angular resolution of measurements recorded by endoscopic probe 10.

In some embodiments, individual insulated stator conductors are stacked at certain circumferential locations on motor 11 (for example, at certain points on housing 28). The stacked stator conductors may be arranged so that the direction of current flow in each set of stacked stator conductors is the same. Therefore, magnetic fields produced by currents flowing in the stacked stator conductors add together. Consequently the magnitude of the magnetic field produced to drive rotor 12 may be increased with the number of stacked conductors (for a given magnitude of current flowing in the stacked stator conductors). In some embodiments, the number of stacked conductors are two or more. In some embodiments the stacked stator conductors are electrically connected in series with one another. In some embodiments the stacked stator conductors are electrically connected together to form a single circuit having one inlet terminal and one outlet terminal such that an electrical current flows through all of each set of stacked stator conductors in the same direction when a single current source is connected at the inlet and outlet terminals to deliver a current through the circuit that includes the stacked stator conductors. In some embodiments plural sets of stacked stator conductors are spaced apart around housing 28. The stacked stator conductors may be connected such that the direction of current flow in one of the sets of stacked stator conductors is opposite to the direction of current flow in another one of the sets of stacked stator conductors.

Different configurations and numbers of stator conductors 26 are possible for producing rotation of rotor 12. For example, some embodiments provide a configuration of six stator conductors 26 connected in three pairs with the stator conductors 26 being spaced apart around housing 28 and driven according to a three-phase scheme.

A driver for motor 11 may have any suitable construction. For example, a timing module (not shown) may be operatively connected to each pair of stator conductors 26. Each timing unit may be configured to control the direction and amount of driving current passing through the corresponding pair of stator conductors 26 at a given time. The timing module may comprise any appropriate controller, such as a microprocessor or other programmable electronics such as a Field Programmable Gate Array. In some embodiments, a pulse width modulation scheme is used to control the current applied to stator conductors 26.

A sheath 28A provides a sterilisable cover for endoscopic probe 10. Sheath 28A may be constructed from any suitable biocompatible material(s). Sheath 28A may be highly flexible for most of its length to allow endoscopic probe 10 to follow turns of small passages. A portion of sheath 28A adjacent to motor 11 is preferably stiff and non-compressible to provide a space in which rotor 12 can rotate freely. Such stiffness may be provided by a housing 28 inside sheath 28A or by making sheath 28A to have a stiff portion that optionally also serves as a housing 28 for motor 11. Sheath 28A may be constructed from any of a wide range of materials including suitable plastics that are sterilisable and otherwise suitable for the application.

Endoscopic probe 10 further comprises a window 30. Window 30 may be formed from any biocompatible material that permits unobstructed travel of light 18 and light 22 to and from tissue 20. The material of window 30 is selected to be compatible with the imaging technique(s) for which probe 10 is intended. In embodiments where Raman spectroscopy is employed, window 30 is preferably constructed from material that is substantially transparent at both a wavelength used to excite Raman spectra and to the resulting Raman spectra such as fluorinated ethylene propylene (FEP), for example. In some embodiments, housing 28 and window 30 are a unitary component.

Endoscopic probe 10 may be used for tissue imaging and/or analysis. In an example application, an endoscopic probe 10 is used by guiding the endoscopic probe 10 to a desired location in a patient's anatomy. For example endoscopic probe 10 may be placed at a desired location in a passage in a patient's body using an endoscopic probe. With motor 11 running, endoscopic probe 10 may be withdrawn along the passage while returning light 22 is collected by endoscopic probe 10 and analyzed by a suitable analysis system or systems (e.g. a spectrometer, Raman spectrometer, OCT system, photomultiplier, and/or photocell). The result can be a helical scan of the tissue surrounding the passage using one or more imaging and/or analysis modalities.

In some example applications motor 11 rotates light deflector 16 at an angular speed in the range of about 1 RPM to about 4000 RPM depending on the imaging/analysis modality used. In some embodiments endoscopic probe 10 is withdrawn at a speed in the range of about 0.02 mm/s to about 70 mm/s.

A variety of imaging and/or analysis modes are possible. Incident light 18 originates from a light source or light sources appropriate for the selected mode(s). For example, endoscopic probe 10 may be used in conjunction with Raman spectroscopy (RS) techniques. In this example embodiment, incident light 18 comprises a laser beam or laser beams in appropriate wavelength ranges, e.g. near ultraviolet, visible, and near infrared regions. When the laser beam(s) illuminate tissue 20, a fraction of the laser beam photons cause tissue molecules to be excited and are scattered with a wavelength that is shifted from the wavelength of the incident beam(s) in a phenomenon known as inelastic scattering. Returning light 22 comprises inelastically scattered photons that are collected and passed through endoscopic probe 10 to a detector.

As another example, endoscopic probe 10 may be used in conjunction with optical coherence tomography (OCT). OCT is based on the principle of interferometry using low-coherence light. Such light may be generated, for example, by superluminescent diodes or pulsed lasers. In some embodiments, light emitted from a light source is split into a scanning and reference light. The scanning light (delivered through endoscopic probe 10 as incident light 18) reflects from tissue 20 and is collected through endoscopic probe 10 as returning light 22. The reference light and the reflected scanning light (e.g. light 22) are allowed to interfere and are detected by a photodetector.

As another example, endoscopic probe 10 may be used in conjunction with fluorescence imaging. In such embodiments, incident light 18 may excite fluorophores in tissue which, in turn emit fluorescence light. The fluorescence light may be returned for analysis as light 22.

In some embodiments, a plurality of imaging techniques are used concurrently or in alternation with endoscopic probes described herein for multi-modal operation.

In some embodiments, endoscopic probes as described herein are used with RS and OCT concurrently. OCT permits imaging over large transverse areas of tissue (>5 mm) with micrometer scale resolution at real-time speed. However, OCT is generally limited to providing reflectivity maps of tissue morphology without providing biochemical specificity. RS offers greater biochemical specificity which can be useful for classifying irregular tissue structures. However, RS suffers from a smaller transverse field of view and a longer integration time than OCT.

The combination of OCT and RS advantageously permits using real-time OCT images to precisely guide RS acquisition, thereby improving overall sampling accuracy. In some embodiments, the operation of such dual-modal imaging techniques comprises sequential transmission and acquisition of RS and OCT signals (e.g. time-division multiplexing). In other embodiments, wavelength-division multiplexing is used for concurrently transmitting and receiving RS and OCT signals on different wavelengths. In some embodiments, endoscopic probes described herein employing RS and/or OCT techniques may be used with white light imaging for general endoscopy and probe navigation purposes.

Employing such techniques using endoscopic probe 10 supports many applications in biomedical and biological fields, such as imaging in neurological, cardiovascular and oncology areas, including diagnosis of early-stage cancers. Returning light 22 carries information on the characteristics of tissue 20, whereby returning light 22 may be analyzed to distinguish between healthy tissue and tumor/cancer tissue, for example.

Endoscopic probe 10 may be inserted into a subject near an organ to be imaged. Insertion of endoscopic probe 10 can be performed by a variety of means such as insertion through the oral cavity or through percutaneous insertion. Endoscopic probe 10 may then be advanced through the internal vessel to a desired imaging site. At the desired location, current may be supplied to stator conductors 26 as described herein to cause rotor 12 and light deflector 16 to rotate about axis 14.

While light detector 16 is being rotated by motor 11, one or more suitable light sources delivers incident light 18 through endoscopic probe 10. As motor 11 turns, the deflected incident light 18′ is emitted in different radial directions. Light 22 from parts of tissue 20 at locations such that the light 22 is captured by light deflector 16 and deflected to travel along endoscopic probe 10 are transmitted back through endoscopic probe 10 to a control module for processing. By operating endoscopic probe 10 in such a manner, a helical or circumferential scan of the tissue lining a passage in a subject may be obtained.

Advantageously, because the electrical components (i.e. stator conductors 26) producing magnetic fields to power rotor 12 do not need to extend as far in the distal direction as light deflector 16, the illustrated design can be implemented without any wires or other obstacles obstructing light or 22. Window 30 can provide an unobstructed 360 degree view of tissues surrounding endoscopic probe 10. This advantage is facilitated by the construction of endoscopic probe 10 which provides a path 15 by way of which at least light 22 can travel through rotor 12 in a direction generally parallel to axis 14.

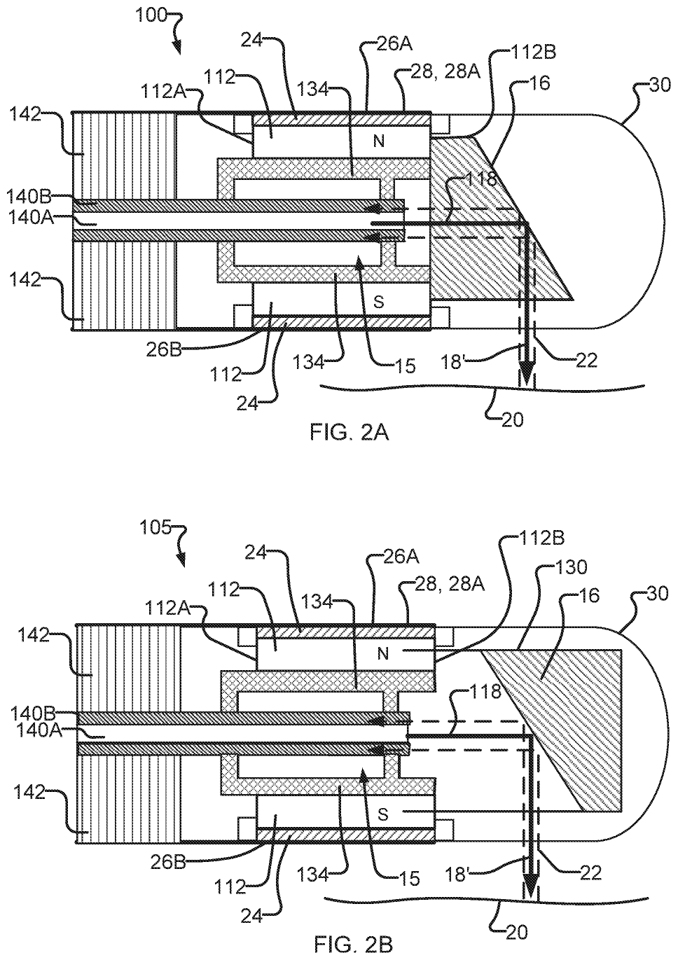

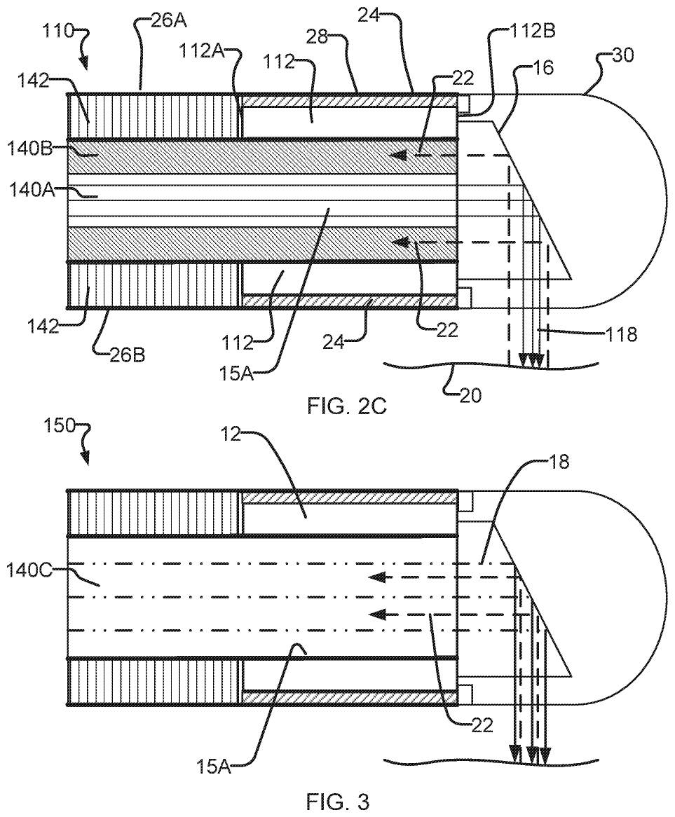

As noted above, it is generally desirable that rotor 12 be physically compact while still containing a sufficient amount of magnetic material to achieve a desired performance of motor 11. This can mean that it is desirable to make the transverse dimensions of any aperture through rotor 12 for path 15 to be small. Endoscopic probe 10 may incorporate various constructions to enhance the efficiency with which light 18 and or light 22 is coupled through path 15. Some examples of such constructions are shown in FIGS. 2 to 15.

Endoscopic probes of the type described herein may be constructed to transmit and detect energy in forms other than light. For example, endoscopic probe 10 may alternatively emit energy in the form of acoustic energy for tissue imaging/analysis. Such embodiments may be constructed generally as described above with light 18 and 22 replaced by another form of energy.

In addition or in the alternative, energy may be carried to or from a location in or at rotor 12 in a different form from the energy that is directed into or received from surrounding tissues. Any appropriate transmission medium may be provided in path 15 to carry energy. For example, a transducer in or near rotor 12 may convert electrical energy into acoustic or light energy or vice versa. The electrical energy may be delivered to and/or received from the transducer by way of wires or other electrical conductors that extend along a probe. In some embodiments the wires extend into an opening that extends axially through rotor 12.

For example, electrical signals may be carried by an electrical cable or conductor disposed along path 15. Similarly, deflector 16 would comprise any appropriate material or element capable of deflecting the particular form of incident and reflected energy travelling in either direction.

One example construction to facilitate coupling of light through rotor 12 is to extend a light guide that carries light along an endoscopic probe to project into an aperture that extends axially along a rotor 12.

FIG. 2A is a schematic longitudinal cross-sectional diagram illustrating a distal end of an endoscopic probe 100 according to an example embodiment. Endoscopic probe 100 operates according to the principle of endoscopic probe 10. Endoscopic probe 100 comprises a light guide 140A operable to carry incident light 118 and a light guide 140B operative to carry returning light 22. In FIG. 2A, light guide 140A is coaxial with and surrounds light guide 140B. The reverse configuration of light guide 140B radially surrounding light guide 140A is possible as is the case where a single light guide is provided. One or both of light guides 140A and 140B extends into an aperture that extends axially along rotor 112. The end of one or both of light guides 140A and 140B is located between ends 112A and 112B of rotor 112. Endoscopic probe 100 comprises a sealing tube 134 which serves to block fluid associated with bearing 24 (e.g. ferrofluid) from reaching light guides 140A, 140B. In the embodiment illustrated in FIG. 2A, sealing tube 134 is coaxial with and surrounds light guides 140A, 140B.

FIG. 2B is a schematic longitudinal cross-sectional diagram illustrating a distal end of an endoscopic probe 105 according to an example embodiment. Endoscopic probe 105 operates according to the principle of endoscopic probe 10. Endoscopic probe 105 comprises a front reflecting light deflector 16 that is supported spaced apart from a distal end of rotor 112 by a holder 130.

FIG. 2C is a schematic longitudinal cross-sectional diagram illustrating a distal end of an endoscopic probe 110 according to an example embodiment. Endoscopic probe 110 operates according to the principle of endoscopic probe 10. Endoscopic probe 100 comprises a light guide 140A operable to carry incident light 118 and a light guide 140B operable to carry returning light 22. In the example of FIG. 2C, light guide 140A is coaxial with and surrounds light guide 140B. The reverse configuration of light guide 140B radially surrounding light guide 140A is possible as is the case where a single light guide is provided. one or both of light guides 140A and 140B extend into an aperture that extends axially along rotor 112. Ends of one or both light guides may be located distally from rotor 112 (i.e. the one or more light guides may extend all of the way through rotor 112) and/or the ends of the light guides may be located between ends 112A and 112B of rotor 112.

Aperture 15A has transverse dimensions sufficient to allow rotor 112 to turn freely while one or both of light guides 140A and 140B extend into aperture 15A defined by rotor 112. In some embodiments, the portions of light guides 140A and 140B proximally adjacent to end 112A and aperture 15A are supported by a support (e.g. support 142 which radially surrounds at least a portion of light guides 140A and 140B). Such a support can serve to support light guides 140A and 140B centered within housing 28 and to aid in directing light guides 140A and 140B into aperture 15A when motor 11 is being assembled. Support 142 may be made from any of a number of suitable materials including metals (e.g. aluminum) or polymers (e.g. FEP and PEBAX). It is desirable for support 142 to be made of a material that is flexible. For example, the use of certain polymers may help enhance the flexibility of endoscopic probe 100 which can be desirable for imaging curved lumens (e.g. the peripheral airways). In some embodiments, a portion of sheath 28A may serve as support 142.

Support 142 may also serve to constrain the axial position of rotor 112. Support 142 may be axially spaced from rotor 112 or be adjacent to rotor 112. In some embodiments, light guides 140A and 140B comprise optical fibers. Light guide 140A may comprise one or more strands of optical fiber for transmitting light 18. Similarly, light guide 140B may comprise one or more strands of optical fiber for receiving light 22. In some embodiments, electrical current is carried to stator conductor 26A, 26B by conductors deposited on the inner wall of support 142 along all or part of the length of endoscopic probe 100.

In the example FIG. 2C embodiment, light guides 140A and 140B are adjacent to or are close to being adjacent to light deflector 16. This feature has the advantage of reducing divergence of incident light(s) 18, increasing the transmission efficiency of endoscopic probe 100. This feature has the further advantage of increasing the efficiency of collection of returning light 22.

FIG. 3 shows an endoscopic probe 150 which is similar in design to endoscopic probe 100. Endoscopic probe 150 comprises a single light guide 140C. Light guide 140C may comprise one or more strands of optical fiber capable of bi-directional communication for transmitting incident light 18 and receiving returning light 22. In some embodiments, transmission of plural light signals through light guide 140C is enabled through wavelength-division multiplexing.

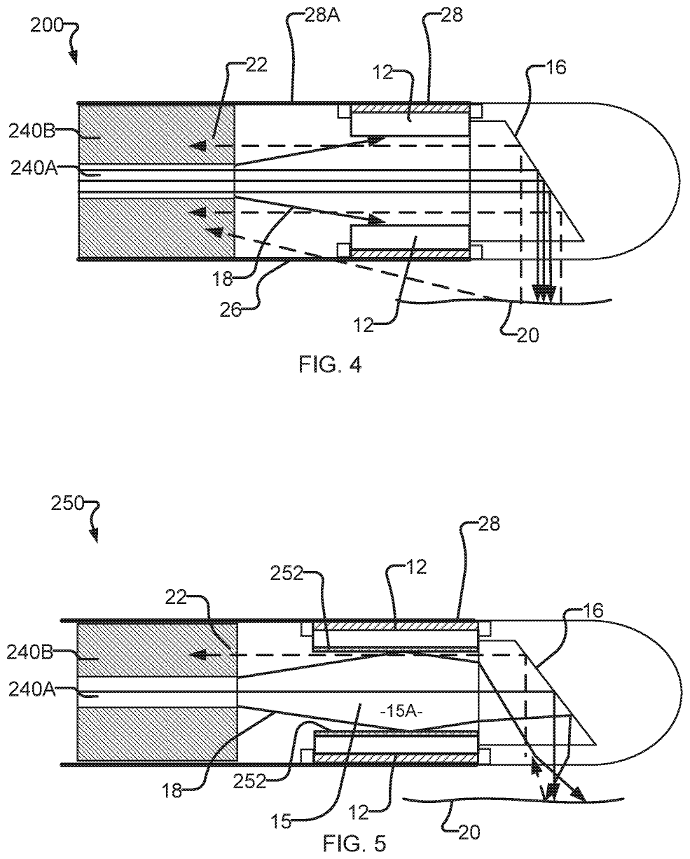

FIG. 4 is a schematic longitudinal cross-sectional diagram illustrating an endoscopic probe 200 according to an example embodiment. Endoscopic probe 200 operates according to the principle of endoscopic probe 10. Endoscopic probe 200 comprises a light guide 240A permitting travel of incident light 18 and a light guide 240B permitting travel of returning light 22. Ends of light guides 240A and 240B are shown to be axially spaced from rotor 212. Compared to endoscopic probes 100 and 150, the design of endoscopic probe 200 permits a smaller diameter of rotor 12 as light guides 240A and 240B do not project into rotor 12.

In some embodiments, sheath 28A of endoscopic probe 200 is transparent in its portion between rotor 12 and the ends of light guides 240A and 240B. In some embodiments, a transparent portion of the sheath comprises FEP. This construction permits the direct collection of returning light 22 that is directed toward the body of endoscopic probe 200 through housing 28, thereby increasing the collection efficiency of endoscopic probe 200. In some embodiments, stator conductors 26 are at least partially transparent for a length of endoscopic probe 200 between the rotor 12 and the distal end of light guide 240B, thus allowing for increased collection of returning light 22. According to an example embodiment, portions of stator conductors 26 are made of indium tin oxide.

FIG. 5 shows an endoscopic probe 250 which is similar in design to endoscopic probe 200 except that endoscopic probe 250 comprises a reflective surface 252 disposed on the interior wall of rotor 12. Reflective surface 252 reflects any light 18 or 22 that reaches reflective surface 252. This reduces the loss of light passing through rotor 12. By contrast, FIG. 4 shows that at least some light that hits the wall of aperture 15 may be absorbed or scattered in such a way that the light is partially lost. This results in a lower transmission efficiency of endoscopic probe 200.

Addition of reflective surface 252 can help to improve the efficiency of light delivery and/or light collection. In some embodiments, reflective surface 252 is obtained by a mechanical and/or chemical polishing process performed on the interior of rotor 12 defining aperture 15A. In some embodiments, reflective surface 252 comprises a coating on the interior wall of rotor 12. Some appropriate materials for the coating include aluminum, chromium, titanium and copper. In other embodiments, reflective surface 252 comprises a thin metallic ring pressed tightly against the interior wall of rotor 12. By employing a separate layer (e.g. a ring) as opposed to a coating, the reflectivity of reflective surface 252 is not affected by the surface quality of the interior of rotor 12.

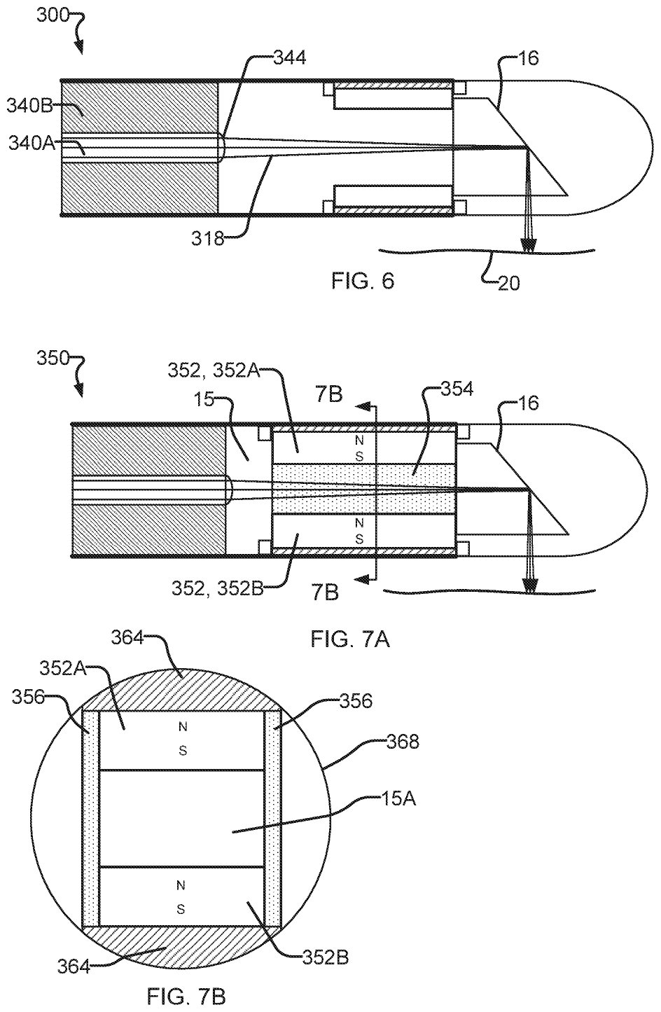

FIG. 6 is a schematic longitudinal cross-sectional diagram illustrating an endoscopic probe 300 according to an example embodiment. Endoscopic probe 300 operates according to the principle of endoscopic probe 10 and comprises a micro-lens 344. Micro-lens 344 is disposed on the end of light guide 340A and is operable to reduce the spread of incident light 18 exiting light guide 340A such that incident light 18 is directed onto a smaller area of light deflector 16.

Focusing incident light 18 advantageously permits a higher proportion of returning light (not shown) to re-enter endoscopic probe 300 and be collected by light guide 340B, thereby increasing transmission efficiency. In some embodiments, micro-lens 344 focuses incident light 18 onto a single point on light deflector 16. Micro-lens 344 may, for example, have one plane surface and one spherical convex surface to refract incident light 18. Fabrication of micro-lens 344 may be performed with direct fabrication methods such as thermal reflow and microdroplet jetting, or with indirect fabrication methods such as machining and MEMS (micro-electromechanical systems) based methods.

FIG. 7A shows an endoscopic probe 350 which is similar in design to endoscopic probe 300. Endoscopic probe 350 comprises a rotor made up of spaced apart joined together thin-plate magnets 352A and 352B. Magnets 352A and 352B are oriented within endoscopic probe 350 such that each magnet 352 has a different pole facing outwardly. In the illustrated embodiment, the north pole of magnet 352A faces the exterior while the south pole of magnet 352B faces the exterior.

FIG. 7B shows an example cross-section view through endoscopic probe 350 in the plane indicated by 7B-7B in FIG. 7A. FIG. 7B illustrates the possibility that rotor 12 may be a composite rotor made of a plurality of magnetic parts joined together. For example, FIG. 7B shows a rotor made of rectangular thin-plate magnets 352A and 352B that are joined together and spaced apart to allow light to pass between them on the way to and from a light deflector 16. Magnets 352A and 352B are shown to be connected together by connector 356.

In some embodiments, connector 356 and tube 368 are constructed from a transparent material. The use of a transparent material permits a portion of light that is reflected toward rotor 352 (as opposed to light deflector 16) to be collected, increasing the efficiency of endoscopic probe 350. The transparent material of connector 356 and tube 368 is preferably selected with reference to the implemented imaging modality (e.g. Raman spectroscopy, visible light, OCT and/or ultrasound). For example, FEP is a suitable material for connector 356 and tube 368 when used in conjunction with Raman spectroscopy. Glass may be a suitable material for connector 356 and tube 368 when visible light or other modalities are used. It is desirable that the transparent material of connector 356 and tube 368 does not add noise to or attenuate incident and reflected light during operation of endoscopic probe 350.

In some embodiments, a light guide is built into and rotates with rotor 12. The light guide may comprise an optical fiber, a collimator, a light integrating rod, a number of lenses or the like that cooperate to carry light through the rotor. In an example embodiment a section of optical fiber that serves as a light guide is disposed within an aperture 15A that extends through a rotor 12. In another embodiment the light guide comprises a transparent window. These constructions can advantageously prevent any fluid (e.g. ferrofluid which may be present) from entering aperture 15 and may additionally enhance the efficiency with which light can be coupled through rotor 12 in one or both directions. This concept is illustrated in FIGS. 7A and 7B by transparent material 354 that fills an aperture in rotor 352. In some embodiments, the transparent material comprises FEP.

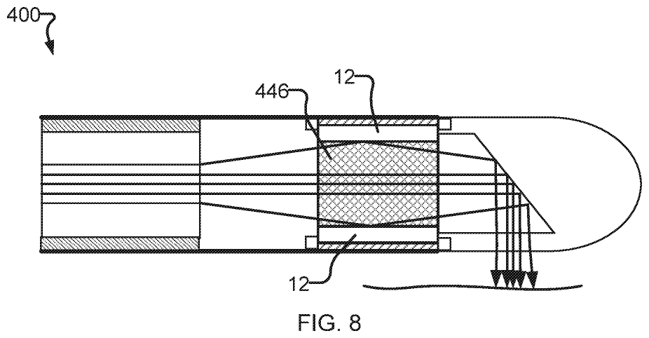

FIG. 8 is a schematic longitudinal cross-sectional diagram illustrating an endoscopic probe 400 according to an example embodiment. Endoscopic probe 400 operates according to the principle of endoscopic probe 10 and comprises a lens 446 disposed within an aperture that extends through rotor 12. In some embodiments, lens 446 comprises a GRIN lens having a material with a refractive index gradient in the radial direction. In other embodiments, lens 446 is a convex lens in any of a variety of possible constructions such as biconvex and plano-convex. Lens 446 is operable to focus transmitted and returning light as shown in FIG. 8, thereby increasing efficiency of transmission of light through rotor 12.

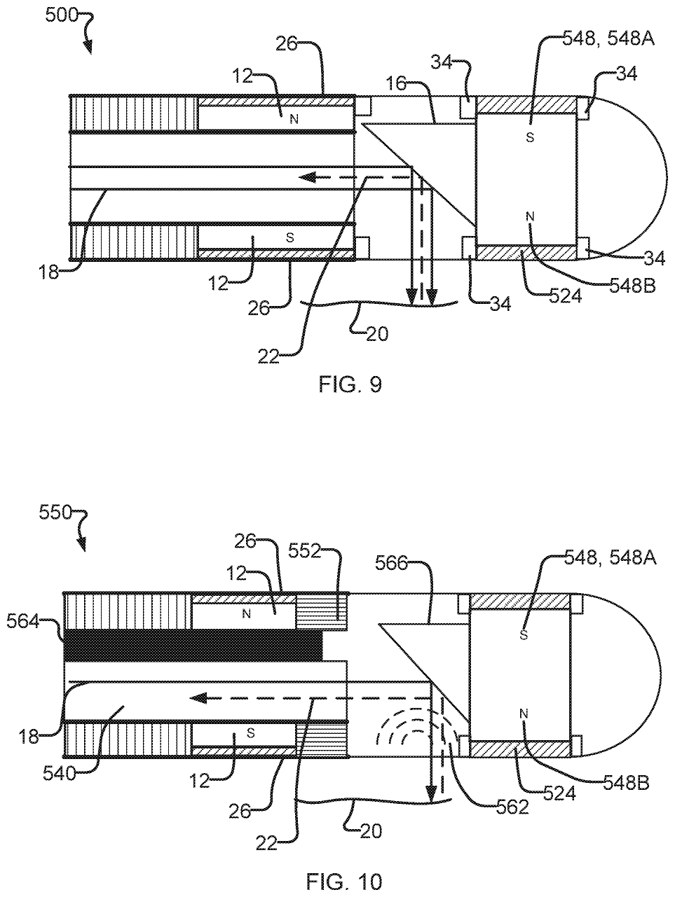

FIG. 9 is a schematic longitudinal cross-sectional diagram illustrating an endoscopic probe 500 according to an example embodiment. Endoscopic probe 500 comprises rotor 12 which operates according to the principle of rotor 12 in endoscopic probe 10. Endoscopic probe 500 comprises a distally located magnet 548 separate from rotor 12. Magnet 548 has opposite magnetic poles 548A and 548B. At its proximal end, magnet 548 is coupled with light deflector 16. Interactions between the magnetic poles of rotor 12 and the magnetic poles of magnet 548 cause magnet 548, and thus light deflector 16, to rotate together with rotor 12. Endoscopic probe 500 further comprises bearing 524 concentrically and circumferentially supporting magnet 548 within endoscopic probe 500. Bearing 524 optionally comprises a ferrofluid bearing as discussed above. As illustrated in the FIG. 9 embodiment, stopper rings 34 may be disposed proximally and distally of magnet 548 in order to constrain axial motion of magnet 548.

FIG. 10 shows an endoscopic probe 550 which is similar in design to endoscopic probe 500. Endoscopic probe 550 operates according to the principles of photoacoustic imaging. In photoacoustic imaging, light pulses delivered into biological tissues are absorbed and converted into heat, resulting in an ultrasonic emission. The generated ultrasound is detected by one or more ultrasound transducers and is processed to produce images of the scanned tissue.

In FIG. 10, incident light 18 produces returning light 22 and ultrasound 562 after incident light 18 interacts with tissue 20. The ultrasound is detected by an ultrasound transducer 552 in endoscopic probe 550. Ultrasound transducer 552 receives ultrasound 562 and converts ultrasound 562 into an electrical signal. In the illustrated embodiment, ultrasound 562 is reflected by deflector 566 toward ultrasound transducer 552.

Endoscopic probe 550 additionally comprises cable 564 connected to carry electrical signals generated by transducer 552 in response to ultrasound 562 to a control module for processing. Endoscopic probe 550 comprises light guide 540 which operates according to the principle of light guide 140C, light guide 540 being capable of bi-directional communication. Light guide 540 functions to transmit incident light 18 and to returning light 22. Similar to rotor 12, ultrasound transducer 552 is hollow or otherwise comprises a cross-sectional shape such that it defines an aperture or path through or into which cable 564 and light guide 540 can extend, allowing light 18 and 22 to be transmitted past ultrasound transducer 552.

In FIG. 10, cable 564 and light guide 540 are shown to be eccentric with one another. In some embodiments, cable 564 is a coaxial cable and light guide 540 comprises one or more strands of optical fiber. In other embodiments, cable 564 and light guide 540 are concentric with one another.

Photoacoustic imaging can provide advantages over other imaging modalities. The non-ionization nature of light waves used in photoacoustic imaging makes it well suited for frequent, repeated use in biological tissues. Photoacoustic imaging is also capable of real-time imaging. The above advantages as well as the well-established, portable, and cost-effective nature of ultrasound imaging devices make photoacoustic imaging especially suitable for continuous and repetitive imaging of disease sites for long-term monitoring of disease progression and/or therapeutic outcome. As a more specific example, due to the above advantages, photoacoustic imaging may be applied for investigations such as monitoring blood flow speed, detecting the blood oxygen level of tumors, producing angiograms of tumor vasculature networks, and tracing molecular probes to visualize the molecular pathology of cancer in vivo.

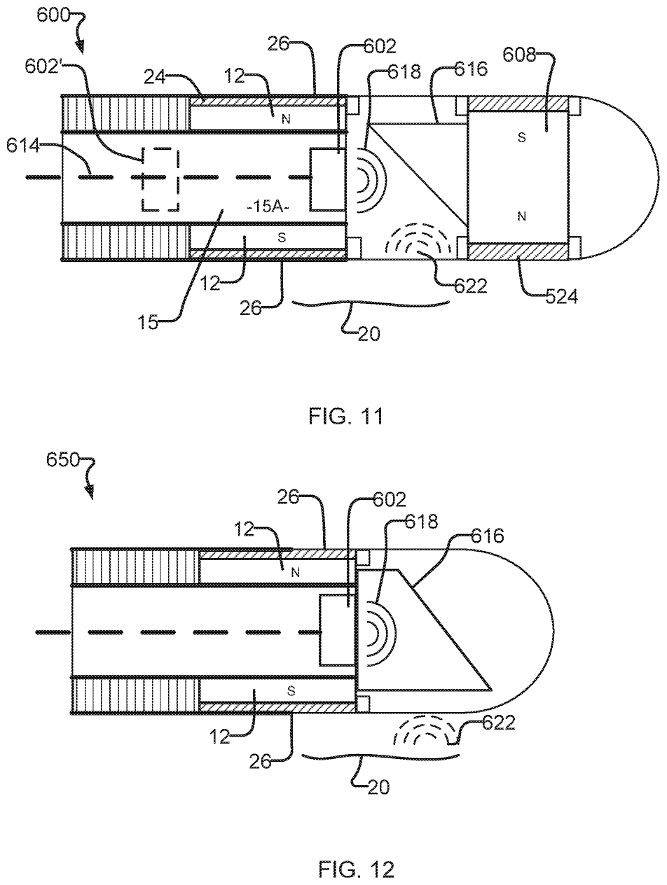

FIGS. 11 to 13 illustrate example embodiments wherein an electronic device such as an ultrasound transducer, light source, and/or light detector (e.g. camera) is supported on a cantilever member that projects into an aperture of a rotor. The electronic device is entirely inside the rotor in some embodiments. The electronic device is located between the rotor and the light detector in some embodiments.

FIG. 11 is a schematic longitudinal cross-sectional diagram illustrating an endoscopic probe 600 according to an example embodiment. Similar to endoscopic probe 500, endoscopic probe 600 comprises a distally located magnet 608 separate from rotor 12. Deflector 616 is coupled to magnet 608. Deflector 616 is driven to rotate by rotor 12 by way of magnetic linkage between magnet 608 and rotor 12.

Endoscopic probe 600 operates according to the principles of ultrasound imaging and comprises an ultrasound transducer 602. In the illustrated embodiment, transducer 602 is supported within axial aperture 15A of rotor 12 by a cantilever member. In other embodiments, transducer 602 may be supported proximally from rotor 12 (e.g. at location 602′) such that ultrasound waves emitted by transducer 602 travel through aperture 15A. Rotor 12 rotates relative to transducer 602. Ultrasound transducer 602 is operable to transmit incident ultrasound 618 which is directed by deflector 616 toward tissue 20 in a direction determined by the current angle of rotation of deflector 616. In some embodiments, ultrasound transducer 602 comprises a plurality of ultrasound transducer elements.

Ultrasound echoes 622 are generated by the interaction of incident ultrasound 618 with tissue. Ultrasound echoes 622 are detected by an ultrasound transducer in probe 600 and are converted into electrical signals. In FIG. 11, transducer 602 both generates ultrasound 618 and detects echoes 622. In the illustrated embodiment, ultrasound echoes 622 are deflected by deflector 616 toward ultrasound transducer 602 Endoscopic probe 600 additionally comprises cable 614 for communicating the electrical signals generated by transducer 602 to a control module for image processing. Cable 614 may also serve as a cantilever member which provides mechanical support to maintain ultrasound transducer 602 at a desired a radial and axial position within aperture 15A. Cable 614 is also operable to deliver electrical signals from the control module to supply power to ultrasound transducer 602 to transmit incident ultrasound 618. In some embodiments, the space surrounding magnet 608 and deflector 616 is filled with a low-viscosity liquid which provides a medium for facilitating efficient travel of ultrasound waves 618 and 622 while permitting magnet 608 (together with deflector 616) and rotor 12 to rotate smoothly. As an illustrative example, pure water can serve as the liquid medium.

Advantageously, where an oil-based ferrofluid is used in ferrofluid bearings 24 and 524 an aqueous medium such as water is immiscible with the ferrofluid and can help to prevent migration of the ferrofluid.

FIG. 12 shows an endoscopic probe 650 which is similar in design to endoscopic probe 600, also employing ultrasound imaging. Endoscopic probe 650 comprises a deflector 616 that is coupled to be driven directly by rotor 12. Probe 650 can otherwise operate in the same manner as probe 600.

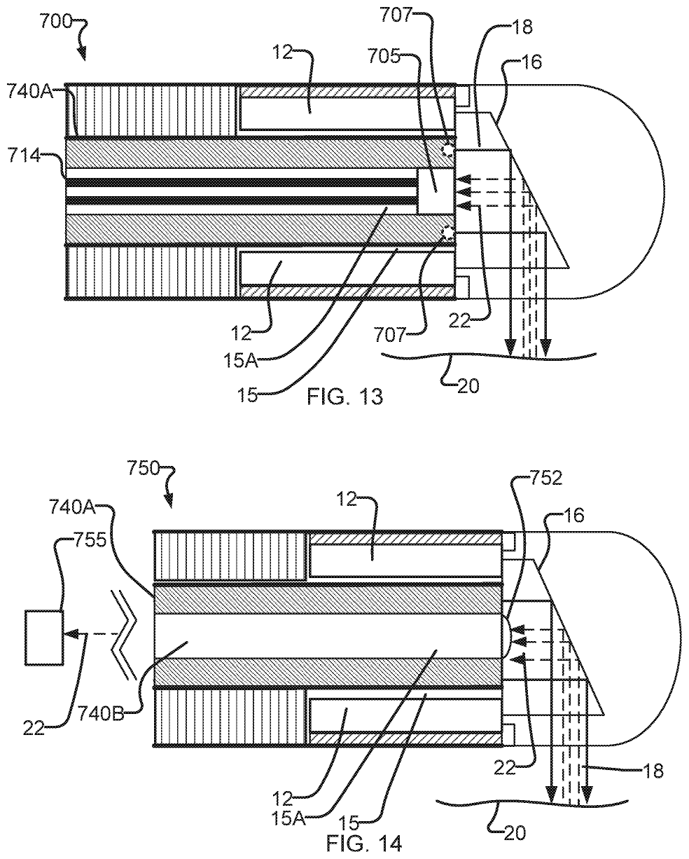

FIG. 13 is a schematic longitudinal cross-sectional diagram illustrating an endoscopic probe 700 according to an example embodiment. Endoscopic probe 700 is similar to endoscopic probe 10 with the addition of an optical sensor 705. Optical sensor 705 is supported by a cable assembly 714 that projects into axial aperture 15 of rotor 12. This construction allows optical sensor 705 to be positioned at or near the distal end of rotor 12.

Endoscopic probe 700 comprises a light guide 740A arranged to carry incident light 18. Returning light 22 reaches optical sensor 705 by way of deflector 16. Returning light 22 is converted into electrical signals by sensor 705. The electrical signals are delivered to a control module by cable 714. Optical sensor 705 optionally comprises plural light sensitive areas. For example, optical sensor 705 may comprise a one dimensional or two dimensional array of light sensitive areas. In some embodiments, image sensor 705 comprises a camera, for example a CCD camera.

In some embodiments, one or more electrically powered light sources 707, such as LEDs, are accommodated radially adjacent to optical sensor 705 in order to deliver incident light 18. Light sources 707 may replace light guide 740A. Such light sources may be powered through cable 714 which delivers power to optical sensor 705, or separate wires or cables (not shown). In some embodiments, optical sensor 705 and/or light guide 740A (or any alternative sources of light) are disposed proximally of rotor 12. This construction allows aperture 15A and hence, rotor 12, to be made smaller. In such embodiments, a collimating lens may be disposed distally of the light source and/or aperture 15A may be filled with transparent material or a lens to avoid unwanted scattering of light (as in the example embodiments shown in FIGS. 7A and 8).

FIG. 14 shows an endoscopic probe 750 which is similar in design to endoscopic probe 700 except that optical sensor 755 is located proximally relative to, and located a distance away from, rotor 12. In probe 750, returning light 22 is directed to optical sensor 755 by way of one or more optical elements which extend into or through axial aperture 15 of rotor 12. In FIG. 14 the optical elements include a lens 752 and a light guide 740B. Optionally, lens 752 may be a magnifying lens which refracts and focuses returning light 22 onto optical sensor 755. Like optical sensor 705, optical sensor 755 may comprise plural light sensitive areas. For example, optical sensor 755 may comprise a 1 dimensional or two dimensional array of light sensitive areas. In some embodiments, image sensor 755 comprises a camera, for example a CCD camera. In an alternative embodiment, light guides 740A and 740B (and the attached lens 752) of the FIG. 14 embodiment do not extend into aperture 15A, but are instead disposed proximally from rotor 12. In such embodiments, aperture 15A may be filled with transparent material or a lens to reduce or avoid unwanted scattering of light.

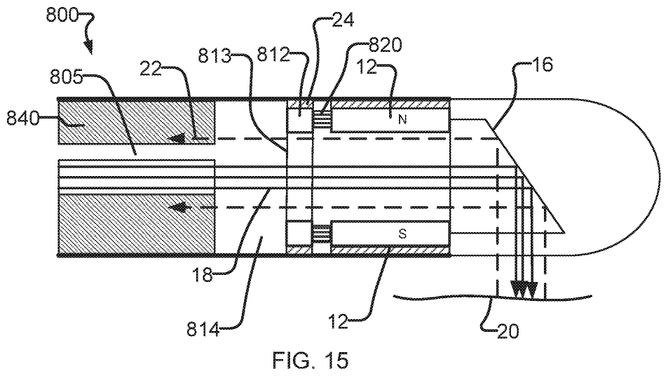

FIG. 15 is a schematic longitudinal cross-sectional diagram illustrating an endoscopic probe 800 according to an example embodiment. Endoscopic probe 800 is similar in principle to endoscopic probe 10 with the addition of a mechanism operable to adjust the axial position of deflector 16. This mechanism may be operated to axially scan surrounding tissue over a limited range without moving probe 800. Combined axial actuation and rotation of rotor 12 can permit a 2D scan or a 3D volumetric scan of tissue to be obtained without having to withdraw or advance endoscopic probe 800 along the passage it is located in.

Endoscopic probe 800 comprises a ring 812 that carries a window 813, which is transparent to light 18 and 22, and is axially movable along probe 800. Rotor 12 is attracted to ring 812. Ring 812 may, for example comprise a ferromagnetic material to which rotor 12 is attracted or an axially magnetized ring-shaped magnet. Ring 812 is supported in a way that allows axial movement with low friction. For example, ring 812 may be supported by a bearing 24 comprising a layer of ferrofluid.

A chamber 814 is defined between the distal end of light guide 840 and ring 812. Chamber 814 is filled with a suitable fluid (e.g. a liquid or gas that is substantially transparent to light 18 and 22, such as pure water or air). Where an endoscopic probe implements an imaging modality that employs a form of energy other than light, the fluid may be selected to be transmissive to that particular form of energy. The axial position of ring 812 may be adjusted by introducing more fluid into chamber 814 or withdrawing some fluid from chamber 814 by way of actuation channel 805.

For example, delivering fluid through channel 805 causes ring 812 to move in a distal direction and withdrawing fluid through channel 805 causes ring 812 to move in a proximal direction. The position of the rotor components (comprising ring 812 and rotor 12 adjoined by mechanical coupler 820) can be controlled by adjusting the amount of fluid being delivered into or being withdrawn from chamber 814. Using an incompressible fluid (i.e. a liquid) in conjunction with this principle of actuation can allow precise control of the position of the rotor components.

Adjusting the axial position of the rotor components also adjusts the axial position of light deflector 16. In the illustrated embodiment, a ferrofluid bearing 24 is magnetically attracted to ring 812. Ferrofluid bearing 24 and window 813 act in conjunction to seal the rotor components against a proximal portion of chamber 814 (which may contain fluid). Ring 812 and rotor 12 are coupled to one another by mechanical coupler 820 such that rotational motion and/or axial linear motion are transmitted between these components by coupler 820 when motion is induced by electromagnetic and/or hydraulic/pneumatic means. Mechanical coupler 820 may also serve to maintain a desired spacing between rotor 12 and ring 812. Mechanical coupler 820 may be formed, for example, by material such as a suitable plastic, metal or ceramic or combinations of these.

In an alternative embodiment, ring 812 and coupler 820 are omitted from the example endoscopic probe 800 illustrated in FIG. 15, This may be done to make the design of endoscopic probe 800 simpler while still enabling axial actuation of rotor 12 based on controlling a volume of fluid in chamber 814. This may be accomplished by providing a radial seal around rotor 12 (such as a ferrofluid bearing 24) and fixing window 813 on a proximal end of rotor 12 to seal rotor 12 against fluid present in chamber 814.

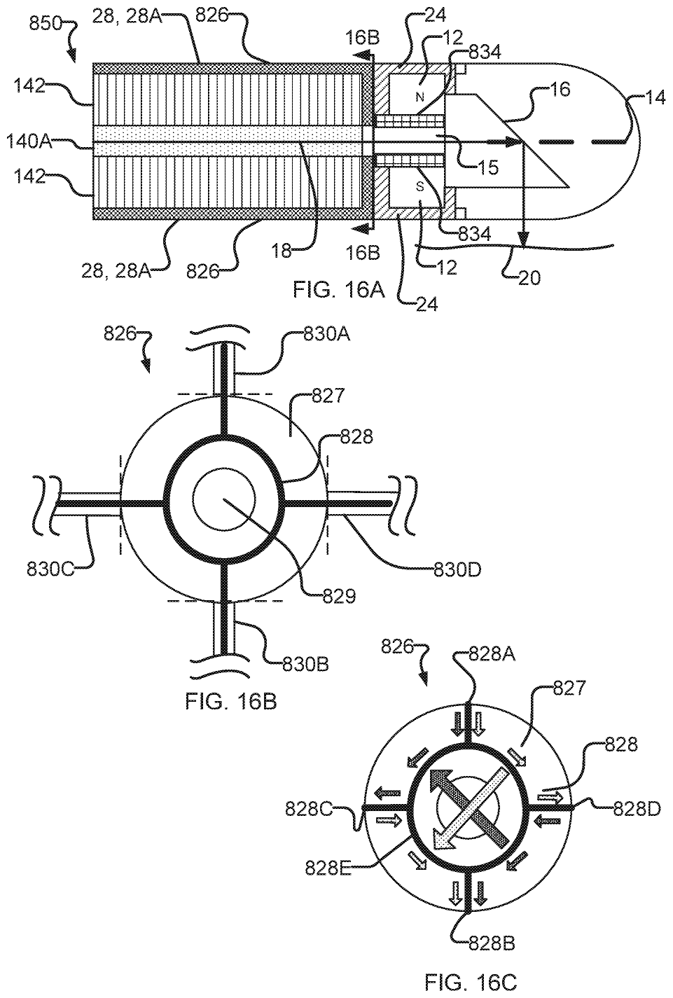

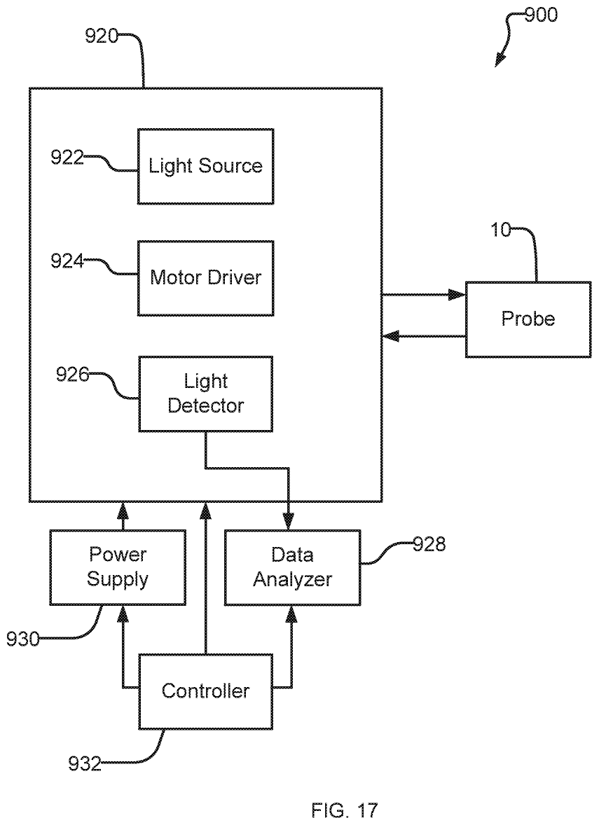

FIG. 16A is a schematic longitudinal cross-sectional drawing of a distal end portion of an example endoscopic probe 850 that includes a planar arrangement of stator conductors that are located radially relative to rotor 12. Endoscopic probe 850 comprises a stator member 826 that comprises a sheet of material 827 that carries electrically conductive traces 828. FIG. 16B is a cross section view of a probe 850 in the plane indicated by line 16B-16B of FIG. 16A. Stator member 826 is located axially adjacent to rotor 12. In this example, stator member 826 is located between support member 142 and stator 12.

FIG. 16B shows that stator member 826 is formed with an aperture 829 that corresponds to optical path 15. Traces 828 include traces 828A, 828B, 828C and 828D that extend inwardly to a circumferential trace 828E. The pattern of traces 828 shown in FIG. 16B is one example pattern. Other patterns for traces 828 are possible.

Electromagnetic fields oriented in different directions may be caused by applying electrical potentials between different combinations of traces 828A, 828B, 828C and 828D. For example: the sequence of electrical potential combinations indicated in the Table 1 will result in a magnetic field that rotates clockwise in 90 degree increments at the location of rotor 12. In Table 1, (+) indicates a first electrical potential and (−) indicates a second electrical potential that is lower than the first electrical potential.

TABLE 1
Example Drive Sequence

| Trace: | 828A | 828B | 828C | 828D |
|---|---|---|---|---|
| Step 1 | (−) | (+) | (−) | (+) |
| Step 2 | (−) | (+) | (+) | (−) |
| Step 3 | (+) | (−) | (+) | (−) |
| Step 4 | (+) | (−) | (−) | (+) |

FIG. 16C is a schematic view of the flow of electrical current (small arrows) and the resultant magnetic fields (large arrows) in stator member 826 for Step 3 (lighter arrows) and Step 4 (darker arrows).

In some embodiments, traces 828A, 828B, 828C and 828D respectively continue along arms 830A, 830B, 830C and 830D (collectively arms 830) that extend from edges of stator member 826. Arms 830 may be folded at 90 degrees to stator member 826 to extend along endoscope probe 850 in a distal direction. A motor driver (not shown) may be connected to traces 828A, 828B, 828C and 828D. The motor driver may apply appropriate sequences of potentials (e.g. as in Table 1) to traces 828A, 828B, 828C and 828D to cause rotor 12 to rotate at a desired rate.

Endoscopic probe 850 of FIG. 16A may comprise a ferrofluid stopper 834 that extends axially from rotor 12 toward stator member 826 and thereby helps to prevent fluid from bearing 24 from entering path 15.

FIG. 17 shows a system 900 comprising an endoscopic probe 10 constructed as described herein. A support system or control module 920 located at a proximal end of endoscopic probe 10 comprises one or more light sources 922 connected to deliver light into endoscopic probe 10, a motor driver 924 connected to apply electrical signals to drive motor 11, one or more light detectors 926 that detect light returned by endoscopic probe 10, a data processing system 928 connected to analyze data received at light detector(s) 926, a power supply 930 for delivering power to support system 920, and a controller 932 providing overall control over the operation of system 900.

System 900 may be implemented in a wide number of ways. In an example embodiment, motor driver 924 comprises a motor driver sub-module comprising an independent power supply, a motor shield, and a controller in communication with controller 932. As an illustrative example, in embodiments where Raman spectroscopy is employed, light source 922 may comprise a diode laser and light detector 926 may comprise a spectrometer and/or spectrograph. In some embodiments, light detector 926 may comprise a camera (e.g. a CCD camera).

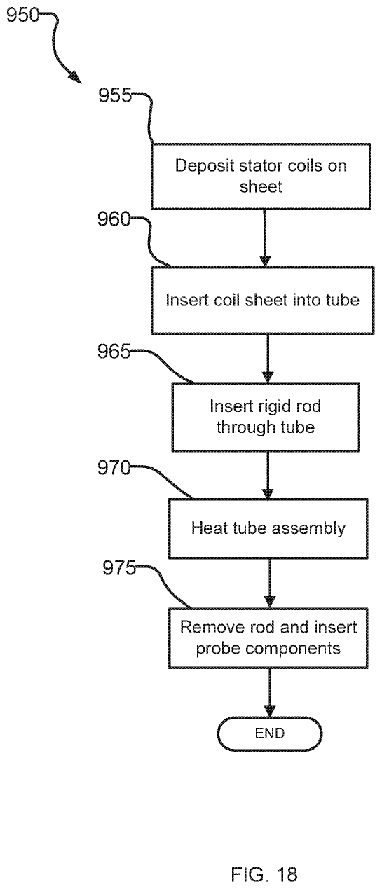

FIG. 18 is flowchart showing a non-limiting example method 950 that can be performed to fabricate endoscopic probes described herein. At step 955, stator conductors are deposited on a flexible sheet, for example, on a polyimide sheet. At step 960, the flexible sheet is rolled and inserted into a heat-shrinkable tube. The material of the heat-shrinkable tube can be selected to enhance the operation of the endoscopic probe based on the selected imaging modality. For example, FEP provides not only optical transparency but is also transparent for Raman spectra when employing Raman spectroscopy. At step 965, a rigid rod with the desired dimensions of the endoscopic probe (i.e. having a diameter equal to the light guide and/or a diameter close to, but slightly larger than, the magnetic rotor to accommodate a bearing) is inserted into the tube, passing through both the folded coil sheet and the tube.

At step 970, the entire assembly comprising the tube, coil sheet and the rigid rod are exposed to heat. Consequently, the tube shrinks to the diameter of the rigid rod and the inner surface of the tube attaches to the stator conductors and the rigid rod with the desired dimensions. At step 975, after the assembly has cooled, the rod is removed to leave the tube with attached stator conductors. The light guide of a selected endoscopic probe may then be inserted into one end of the tube. Also at step 975, a motor (comprising a rotor and a bearing) coupled with a light deflector of the endoscopic probe may be inserted into the tube at the other end, together with a window or capping component to complete assembly of the endoscopic probe.

By employing a fabrication technique such as that shown in method 950, endoscopic probes comprising a motor and a light guide can be assembled into the same tube, with the probe components being aligned concentrically with high precision. This fabrication technique, through the self-aligning of components, enables high-precision optical alignment between the light guide, the light deflector coupled to the rotor, and any other optical elements which are present (e.g. a micro-lens at a distal end of the light guide). Other methods and variations of fabricating endoscopic probes are possible to achieve the self-alignment of various components. For example, the rigid tube of method 950 may be omitted and the heat-shrinkable tube may be directly shrunk onto the outer walls of a motor housing and the outer walls of a light guide. In an alternative embodiment, a motor and a light guide can be tight fit into opposite ends of a fixed diameter tube with outer diameters of the motor and light guide being selected to match the inner diameter of the tube.

An alternative method of assembling endoscopic probes comprises providing a cylindrical rotor coated with a removable outer layer of uniform thickness. A heat shrinkable tube containing stator conductors may be positioned around the pre-coated rotor (as in step 965 of method 950) and subsequently, the assembly may be exposed to heat to shrink the tube onto the rotor (as in step 970). The removable layer is then selectively dissolved or otherwise removed in a wet or dry etchant. Subsequently, a ferrofluid is injected into the space created between the inner wall of the tube and the outer wall of the rotor to provide a ferrofluid bearing. In other embodiments, a rotor having a rectangular cross section is used and the coating of a removable outer layer onto the rotor results in the rotor and the outer layer having a circular cross-section.

Features of the embodiments discussed herein may be combined. For example, the feature of reflective surface 252 of FIG. 5 may be combined with the feature of micro lens 344 of FIG. 6.

Advantageously, an endoscopic probe as described herein may be made to have a small diameter. For example, a diameter of a motor 11 may be 2 mm or less. An outside diameter in a section of the endoscopic probe extending from the distal end is for a desired distance such as at least 50 cm or at least 1 m or more toward the proximal end may be 2 mm or less.

The present technology may provide various advantages as compared to other endoscopic probes. Some embodiments may provide one or more of the following advantages:
Endoscopic probes can have a relatively short stiff section in the vicinity of the motor. Other portions of the probes may be flexible. This may allow the probes to be used in small diameter tortuous passages.
Endoscopic probes may maximize emission and collection of light, ultrasound or other forms of energy through transparent windows with no obstruction from opaque objects such as electrical wires, thus enabling unobstructed 360 degree scanning.
Short length of a motor/deflector can be facilitated by using a magnetic rotor having an axial aperture such that the rotor and a wave transmission medium, transducers or other components may occupy the same axial space within a probe and/or supporting the magnetic rotor in a low friction bearing such that a shorter rotor can develop sufficient torque to rotate a deflector at a desired angular speed.
Micromotors of the present invention can be operated at large range of speeds. The range may include operating the micromotor in a mode comprising angle-resolved stepping at arbitrary angles, or alternatively, in a mode is rotated at a high speed. This can be accomplished by varying the amplitudes and timing of driving currents supplied to stator conductors of the motor.
The designs are compatible with the use of low friction ferrofluid bearings. The self-sustaining quality of ferrofluid bearings significantly simplifies the design of bearings and potentially lowers cost by eliminating the need for any precision alignment and/or special assembly to establish the bearing of the rotor.
The self-aligned assembly process enables higher optical transmission through the probe through its ability to attain high-precision optical alignment of elements. Additionally, due to the simplicity of the described process, costs can be lowered in the fabrication of endoscopic probes.

It can be appreciated that the teaching provided herein may be modified in many ways and applied to create other example embodiments. For example, the disclosed principle of providing a micromotor having a rotor which includes an axial aperture may be applied to micromotors which operate on other principles. For example, piezoelectric or ultrasonic micromotors may be employed in place of the electromagnetic motors described herein.

Interpretation of Terms

Unless the context clearly requires otherwise, throughout the description and the claims:
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms;
“distal” means a direction toward the free end of an endoscopic probe along the length of the endoscopic probe (e.g. in the described embodiments motor 11 is located at or near a distal end of the described probes); and
“proximal” means a direction along an endoscopic probe toward an end of the endoscopic probe that connects to a control module or support system (e.g. an end of an endoscopic probe that normally remains outside of a patient).

Words that indicate directions relative to an apparatus device or system such as “vertical”, “transverse”, “horizontal”, “upward”, “downward”, “forward”, “backward”, “inward”, “outward”, “vertical”, “transverse”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

Where a component (e.g. endoscopic probe, mirror, deflector, rotor, bearing, stator conductors, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

Various features are described herein as being present in “some embodiments”. Such features are not mandatory and may not be present in all embodiments. Embodiments of the invention may include zero, any one or any combination of two or more of such features. This is limited only to the extent that certain ones of such features are incompatible with other ones of such features in the sense that it would be impossible for a person of ordinary skill in the art to construct a practical embodiment that combines such incompatible features. Consequently, the description that “some embodiments” possess feature A and “some embodiments” possess feature B should be interpreted as an express indication that the inventors also contemplate embodiments which combine features A and B even if the descriptions of features A and B are not located together (unless the description states otherwise or features A and B are fundamentally incompatible).

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

1. An endoscopic probe comprising:
a flexible light guide extending from a proximal end of the endoscopic probe to a distal end portion of the endoscopic probe;
a motor in the distal end portion of the endoscopic probe, the motor comprising a rotor that is rotatable relative to the light guide about an axis of rotation;
a light deflector connected to be driven to rotate by the rotor, the light deflector located at a location between the rotor and a distal end of the endoscopic probe;
wherein the rotor is configured to provide a light path extending axially through the rotor, the light path arranged to carry light between the light deflector and the light guide;
wherein the light deflector is surrounded by an unobstructed window that extends 360 degree around the axis and the deflector at the location between the rotor and the distal end of the endoscopic probe.

2. The endoscopic probe according to claim 1 wherein the light path comprises an aperture that extends axially into the rotor.

3. The endoscopic probe according to claim 1 wherein the light guide extends into the rotor along the light path.

4. The endoscopic probe according to claim 2 comprising a cantilever member extending into the aperture and an electronic device supported by the cantilever member.

5. The endoscopic probe according to claim 2 comprising a lens supported in the light path to rotate with the rotor.

6. The endoscopic probe according to claim 1 comprising a light guide in the light path and coupled to rotate with the rotor.

7. The endoscopic probe according to claim 1 comprising an optically transparent window carried on the rotor.

8. The endoscopic probe according to claim 1 wherein the light deflector comprises a mirror or a prism.

9. The endoscopic probe according to claim 1 wherein the light deflector is coupled to be rotated by the rotor by magnetic interaction between the rotor and a magnet attached to the light deflector.

10. The endoscopic probe according to claim 1 wherein the light deflector is coupled to be rotated by the rotor by a member that attaches the light deflector to the rotor.

11. The endoscopic probe according to claim 1 comprising a micro-lens disposed at a distal end of the light guide.

12. The endoscopic probe according to claim 1 wherein the rotor comprises a magnet.

13. The endoscopic probe according to claim 1 wherein the rotor consists of a permanent magnet, with north and south magnetic poles on first and second opposed axially-extending surfaces of the magnet and a hole extending axially between the first and second surfaces through the permanent magnet from a proximal end face of the magnet to a distal end face of the magnet wherein the light path extends along the hole.

14. The endoscopic probe according to claim 1 comprising a fluidic channel extending from the proximal end of the endoscopic probe to a chamber located adjacent to the rotor wherein the rotor is supported for axial movement relative to the light guide and an axial position of the light deflector is adjustable by introducing a fluid into the chamber by way of the fluidic channel or withdrawing the fluid from the chamber by way of the fluidic channel.

15. The endoscopic probe according to claim 1 wherein the rotor is radially supported in an interior of a tubular motor housing by one or more bearings.

16. The endoscopic probe according to claim 15 wherein the motor is magnetic and the one or more bearings comprise ferrofluid bearings.

17. The endoscopic probe according to claim 15 comprising one or more stoppers arranged to axially support the rotor wherein the one or more stoppers comprise sealing media operable to maintain an axial position of the one or more bearings.

18. The endoscopic probe according to claim 1 comprising plural stator conductors extending along the endoscopic probe toward the distal end of the endoscopic probe, the stator conductors extending past the rotor in a generally axial direction at locations circumferentially spaced apart around the rotor.

19. The endoscopic probe according to claim 1 comprising one or more stator conductors extending along the endoscopic probe toward the distal end of the endoscopic probe, wherein a portion of the stator conductors extend in a generally radial direction to a corresponding points circumferentially spaced apart around a circumferential conductor that is axially adjacent to the rotor.

20. The endoscopic probe according to claim 18 wherein portions of the stator conductors are provided by traces on a flexible printed circuit sheet that is flexed to curve around the rotor.
